(19) United States
(12) Patent Application Publication
(10) Pub. No.: US 2016/0228537 A1
(43) Pub. Date: Aug. 11, 2016

(54) REVERSE VACCINATION THERAPY OF MULTIPLE SCLEROSIS

(71) Applicant: CREATIVE MEDICAL HEALTH, LLC, Phoenix, AZ (US)

(72) Inventor: Thomas ICHIM, San Diego, CA (US)

(21) Appl. No.: 15/017,381

(22) Filed: Feb. 5, 2016

Related U.S. Application Data
(60) Provisional application No. 62/112,271, filed on Feb. 5, 2015.

(51) Int. Cl. A61K 39/235; A61K 39/21

(52) U.S. Cl. CPC A61K 39/235; A61K 39/21; C12N 2770/00034; C12N 2710/10034

(57) ABSTRACT

Disclosed are means of inducing antigen-specific tolerance through genetically modifying MSC to express antigens of interest in an inducible manner or constitutive manner. MSC have been demonstrated to suppress pathological immunity in an antigen-nonspecific manner in vitro and in vivo, including clinical trials of GVHD, Type 1 Diabetes, and Multiple Sclerosis. Administration of autoantigens in non-immunogenic routes, such as orally, intranasally, or delivered using immature dendritic cells has shown some signs of clinical efficacy, although effect has not been robust enough to allow for human therapeutic success. We disclose genetic modification of MSC to induce overexpression of autoantigens in a regulated manner in order to generate a universal donor antigen-specific tolerogenic vaccine as a treatment for autoimmunity. MSC are uniquely suited for this goal given their following properties: a) induction of T regulatory cells; b) suppression of T helper, T cytotoxic, and NK cells; c) downregulation of antigen presentation.

### CROSS-REFERENCE TO RELATED APPLICATIONS

This application claims the benefit of U.S. Provisional Application No. 62/112,271, filed Feb. 5, 2015, which is hereby incorporated in its entirety including all tables, figures and claims.

### DESCRIPTION OF THE INVENTION

Immunological tolerance is a cardinal feature of the immune system, allowing for recognition and elimination of pathological threats, while selectively ignoring antigens that belong to the body. Understanding mechanisms of immunological tolerance, and having the ability to induce this process would make a major impact in autoimmune conditions, which affect approximately 8% of the US population¹. Major autoimmune diseases include rheumatoid arthritis, multiple sclerosis, type 1 diabetes, systemic lupus erythromatosis, and inflammatory bowel disease. Traditionally, autoimmune conditions are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia. The introduction of “biological therapies” such as anti-TNF-alpha antibodies has led to some improvements in prognosis, although side effects are still present due to the non-specific nature of the intervention. Regardless, sales of TNF-alpha inhibitors have been quite successful: Humira ($9.2B; 2012), Enbrel ($7.8B; 2011), Remicade ($6.7B; 2011)². These approaches do not “cure” autoimmunity, but merely alleviate symptomology. ¹ http://www.niaid.nih.gov/topics/autoimmune/Documents/adccreport.pdf² http://www.forbes.com/sites/simonking/2013/01/28/the-best-selling-drugs-of-all-time-humira-joins-the-elite/

To “cure” autoimmunity, it is essential to delete/inactivate the T cell clone that is recognizing the autoantigen in a selective manner. This would be akin to recapitulating the natural process of tolerance induction. While thymic deletion was the original process identified as being responsible for selectively deleting autoreactive T cells, it became clear that numerous redundant mechanisms exist that are not limited to the neonatal period. Specifically, a “mirror image” immune system was demonstrated to co-exist with the conventional immune system. Conventional T cells are activated by self-antigens to die in the thymus and conventional T cells that are not activated receive a survival signal [1]; the “mirror image”, T regulatory (Treg) cells are actually selected to live by encounter with self-antigens, and Treg cells that do not bind self antigens are deleted [2, 3]. Thus the self-nonself discrimination by the immune system occurs in part based on self antigens depleting autoreactive T cells, while promoting the generation of Treg cells. An important point for development of an antigen-specific tolerogenic vaccine is that in adult life, and in the periphery, autoreactive T cells are “anergized” by presentation of self-antigens in absence of danger signals, and autoreactive Treg are generated in response to self antigens. Although the process of T cell deletion in the thymus is different than induction of T cell anergy, and Treg generation in the thymus, results in a different type of Treg as compared to peripheral induced Treg, in many aspects, the end result of adult tolerogenesis is similar to that which occurs in the neonatal period.

Specific examples of tolerogenesis that occurs in adults includes settings such as pregnancy, cancer, and oral tolerance. In the situation of pregnancy, studies have demonstrated selective inactivation of maternal T cell clones that recognize fetal antigens occurs through a variety of mechanisms, including FasL expression on fetal and placental cells [4], antigen presentation in the context of PD1-L [5], and HLA-G interacting with immune inhibitory receptors such as ILT4 [6]. In pregnancy, “tolerogenic antigen presentation” occurs only through the indirect pathway of antigen presentation [7]. Other pathways of selective tolerogenesis in pregnancy include the stimulation of Treg cells, which have been demonstrated essential for successful pregnancy [8]. In the context of cancer, depletion of tumor specific T cells, while sparing of T cells with specificities to other antigens has been demonstrated by the tumor itself or tumor associated cells [9-12]. Additionally, Treg cells have been demonstrated to actively suppress anti-tumor T cells, perhaps as a “back up” mechanism of tumor immune evasion [13-15]. At a clinical level the ability of tumors to inhibit peripheral T cell activity has been associated in numerous studies with poor prognosis [16-18]. Oral tolerance is the process by which ingested antigens induce generation of antigen-specific TGF-beta producing cells (called “Th3” by some) [19-21], as well as Treg cells [22, 23]. Ingestion of antigen, including the autoantigen collagen II [24], has been shown to induce inhibition of both T and B cell responses in a specific manner [25, 26]. It appears that induction of regulatory cells, as well as deletion/anergy of effector cells is associated with antigen presentation in a tolerogenic manner [27]. Remission of disease in animal models of RA [28], multiple sclerosis [29], and type I diabetes [30], has been reported by oral administration of autoantigens. Furthermore, clinical trials have shown signals of efficacy of oral tolerance in autoimmune diseases such as rheumatoid arthritis [31], autoimmune uveitis [32], and multiple sclerosis [33]. In all of these natural conditions of tolerance, common molecules and mechanisms seem to be operating. Accordingly, a natural means of inducing tolerance would be the administration of a “universal donor” cell with tolerogenic potential that generate molecules similar to those found in physiological conditions of tolerance induction. Below we will describe some of the mechanisms associated with mesenchymal stem cell (MSC) mediated tolerogenesis. While to date, MSC mediated tolerogenesis is non-specific to a particular antigen, we propose that genetic transfection of autoantigens in an inducible manner will allow the use of MSC to act as an “antigen-specific tolerogenic vaccine”.

### Background on Mesenchymal Stem Cells (MSC)

Mesenchymal stem cells (MSC) are classically defined as adherent, non-hematopoietic cells expressing markers such as CD90, CD105, and CD73, while lacking expression of CD14, CD34, and CD45, and being able to differentiate into adipocytes, chondrocytes, and osteocytes in vitro after treatment with differentiation inducing agents [34]. Although early studies in the late 1960s initially identified MSC in the bone marrow [35], more recent studies have reported these cells can be purified from various tissues such as adipose [36], heart [37] , Wharton's Jelly [38], dental pulp [39], peripheral blood [40], cord blood [41], and more recently menstrual blood [42-44]. Studies in the bone marrow showed that although MSC are the primary cell type that overgrows in in vitro cultures, in vivo MSC are found at a low ratio compared to other bone marrow mononuclear cells, specifically, 1:10,000 to 1:100,000 [45]. The physiological role of MSC still remains to be fully elucidated, with one hypothesis being that bone marrow MSC act as precursors for stromal cells that make up the hematopoietic stem cell microenvironment [46-48].

The first clinical use of MSC has been to accelerate hematopoietic recovery after bone marrow ablation in the context of hematopoietic stem cell transplant or oncology chemotherapy. In a 1995 paper, Lazarus et al. reported the use of autologous, in vitro expanded, “mesenchymal progenitor cells” to treat 15 patients suffering from hematological malignancies in remission. The authors demonstrated that a 10 cc bone marrow sample was capable of 16,000-fold growth over a 4-7 week in vitro culture. Cell administration was performed in total doses ranging from 1−50×10⁶ cells and was not causative of treatment associated adverse effects [49]. In a subsequent study from the same group, the use of MSC to accelerate hematopoietic reconstitution was performed in 28 breast cancer patients who received high dose chemotherapy. MSC at concentrations of 1.0−2.2×10⁶/kg, were administered intravenously with no treatment associated adverse effects. The authors noted that leukocytic and thrombocytic reconstitution occurred at an accelerated rate as compared to historical controls [50]. It is important to note that these initial clinical experiences with MSC were in patients with oncological indications and no overt acceleration of cancer progression was noted. This has been a concern given that MSC are known to be angiogenic [51-56], produce mitogenic/antiapoptotic factors [57-63], and exert an immune suppressive effect [64-71]. Besides feasibility, these studies were important because they established the technique for ex vivo expansion and re-administration.

The demonstration of clinical feasibility, combined with animal models supporting therapeutic efficacy of MSC in non-hematopoietic indications [72-79], gave rise to a series of clinical trials with MSC in a wide range of therapeutic areas ranging from major diseases such as stroke [80-83], heart failure [84, 85], COPD [86], and liver failure [87], as well as rare diseases such as osteogenesis imperfecta [88], Hurler syndrome [89], and Duchenne Muscular Dystrophy [90]. The ability to generate large amounts of defined MSC starting with a small clinical sample, to administer without need for haplotype matching, and excellent safety profile, has resulted in a current 367 clinical trials listed on the international registry clinicaltrials.gov. While some trials have demonstrated efficacy of MSC, little is known about molecular mechanisms. Initial studies demonstrated ability of certain MSC types to differentiate into functional tissues that is compromised as a result of the underlying pathological. Subsequent studies have demonstrated that the majority of administered MSC lodge into the lungs and liver, with only a small minority entering the tissue of pathology [91].

### MSC Therapeutic Activity is Stimulated by Physiological Need

The concept that MSC act as “repair cells” of the body, would imply that MSC do not constitutively secrete regenerative factors, but in contrast, produce them only upon need. Indeed this seems to be not just a concept but an experimental reality. One of the most common elements of tissue injury is the presence of hypoxia. Interstitial damage is often associated with activation of the coagulation cascade, resulting in areas of hypoxia. It is known that reduction in oxygen tension in a variety of tissues leads to activation of the transcription factor HIF-1 alpha, which induces transcription of angiogenic genes such as VEGF [92, 93], as well as the MSC chemoattractant SDF-1 [94, 95]. Once MSC migrate to areas of hypoxia, it has been demonstrated that production of various therapeutic paracrine mediators is increased. The relevance of hypoxia to MSC growth factor production in vitro has been demonstrated by several groups. For example, exposure of bone marrow (BM)-MSC to 24 hours of hypoxia (1% oxygen) results in marked induction of VEGF, FGF-2, HGF, and IGF-1 production, in an NF-kappa B dependent manner [96]. The stimulation of growth factor production by hypoxia is not specific to BM-MSC and has been demonstrated in MSC derived from adipose tissue [97], placenta [98], and dental pulp [99]. Furthermore, hypoxia stimulation of angiogenic and anti-apoptotic factors such as VEGF, FGF-2, HGF and IGF-1 have been reported to also occur in MSC from aged animals, supporting clinical utility [100].

The biological relevance of MSC-secreted growth factors stimulated by hypoxia can be seen in studies showing that conditioned media from hypoxia treated MSC but not normoxia treated MSC endows therapeutic benefit in animal models. For example, Chang et al demonstrated that conditioned media from hypoxia treated BM-MSC was capable of restoring neurological function in a rat model of traumatic brain injury significantly better than administration of conditioned media from normoxia treated BM-MSC. Furthermore, they demonstrated that this was associated with production of HGF and VEGF, which were involved in the induction of endogenous neurogenesis [101]. In a similar study, Yu et al administered BM-MSC that were hypoxia conditioned into a rat massive hepatectomy model, and compared their therapeutic activity with BM-MSC that were exposed to normoxia. They found that the hypoxia conditioned BM-MSC produced significantly higher levels of VEGF in vitro as compared to control treated cells. Furthermore, in vivo administration resulted in significantly elevated cyclin D1, proliferating cell nuclear antigen-positive hepatocytes, liver weight/body weight ratio, and survival compared with animals that received normoxia preconditioned BM-MSC. Interestingly, blockade of VEGF by in vivo administration of anti-VEGF antibody negated the therapeutic effect of hypoxia [102]. In a rat model of diabetic cardiomyopathy it was demonstrated that administration of hypoxia treated BM-MSC resulted in superior inhibition of pathological remodeling as compared to administration of control BM-MSC, which was associated with production of cardiomyocytes from apoptosis, which the authors speculated may have occurred due to hypoxia mediated stimulation of IGF-1 production [103].

The effect of hypoxia on MSC appears to be not only a trigger for production of growth factors, but also allows the MSC to retain an undifferentiated phenotype, allowing for self-renewal without differentiation. This may be due in part to the fact that anatomically, MSCs tend to be found in hypoxic areas of the body, for example in areas of adipose tissue, or bone marrow that are relatively poorly perfused by the circulatory system [104, 105]. It was demonstrated in vitro that exposure of BM-MSC to hypoxia results in augmented proliferation of BM-MSC, as well as the formation of colonies in the colony-forming unit assay (CFU-A), the percentage of quiescent cells, and the expression of stemness markers Rex-1 and Oct-4, thereby suggesting an increase in the stemness of BM-MSC when exposed to hypoxia [106].

One of the key factors of MSC of relevance to therapeutics development is their known anti-inflammatory/immune modulatory properties. The potency of this effect is seen in clinical studies showing efficacy of MSC at inhibiting the lethal, immune-based condition of graft versus host disease [107-112]. Exposure of MSC to hypoxia has been demonstrated in several systems to augment immune modulatory activity. In one example, it was demonstrated that MSC expression of the tryptophan catabolizing enzyme, indolamine 2,3 deoxygenase (IDO), was markedly upregulated in the presence of hypoxia [113]. IDO is critical in immune regulation by MSC in part through induction of T cell anergy [114], and in part by stimulation of T regulatory cells [115, 116]. The practical relevance of hypoxia-stimulated immune regulation of MSC is seen in the situation of allogeneic use of BM-MSC for stimulation of therapeutic angiogenesis. It was shown in a recent study that hypoxia-conditioned BM-MSCs from B6 mice ameliorate limb ischaemia of Balb/c mice compared to normoxic MSCs. Histological staining demonstrated that hypoxic BM-MSC have an increased ability to engraft in allogeneic recipients by reducing NK cytotoxicity, and decrease the accumulation of host-derived NK cells when transplanted in vivo. These allogeneic hypoxia treated BM-MSC gave rise to CD31+ endothelial cells and αSMA+ and desmin+ muscle cells, thereby enhancing angiogenesis and restoring muscle structure. Moreover, application of anti-NK antibodies together with normoxic MSCs enhanced angiogenesis and prevented limb amputation in allogeneic recipients with limb ischemia, thus demonstrating that the benefit of hypoxic conditioning was mediated by enhanced immune modulation in the allogenic setting [117].

In addition to responding to hypoxia, MSC produce immune modulatory and regenerative factors in response to inflammatory stimuli. One of the most studied mechanisms by which inflammation triggers MSC activity the treatment with interferon gamma. This cytokine is typically produced during inflammatory Th1 immune responses that are associated with autoimmunity mediated by cellular means, such as CD8 T cells and NK cells. Examples of conditions associated with this type of immune response include multiple sclerosis, type 1 diabetes, and rheumatoid arthritis [118]. Exposure of MSC to interferon gamma has been demonstrated by numerous groups to increase the immune suppressive activity by stimulation of the enzyme IDO [119-122]. As expected, exposure to this inflammatory mediator induces production of other inhibitors of inflammation by MSC, including the complement inhibitor Factor H [123], as well as the immune modulatory molecules TGF-beta and HGF [124]. At a functional level, Noone et al demonstrated that interferon gamma pretreatment of MSC resulted in protection of the MSC from NK-mediated killing in part through upregulation of prostaglandin E (PGE)-2 synthesis [125]. Other inflammatory mediators that are known to induce regenerative activities in MSC include the macrophage-derived cytokine TNF-alpha. It was demonstrated that TNF-alpha pretreated of MSC endowed the cells with superior angiogenic activity in vitro, as assessed by expression of VEGF, as well as in vivo, for treating the animal model of critical limb ischemia, as compared to untreated MSC [126]. Another study demonstrated that TN alpha pre-conditioning increased proliferation, mobilization, and osteogenic differentiation of MSC and up-regulated bone morphogenetic protein-2 (BMP-2) protein level. BMP-2 silencing by siRNA partially inhibited osteogenic differentiation of MSC induced by TNF alpha [127]. More recent studies have shown that activators of innate immunity, such as lipopolysaccharide, and TLR agonists, also are capable of stimulating regenerative activity in MSC through production of paracrine factors such as VEGF [128].

Overall, these data suggest that the paracrine effects of MSC seem to be inducible, and have a relationship with context-specific settings. Specifically, the role of the MSC is to act as a “repair cell” but it only performs these functions in the context in which it is needed to perform this.

### Intravenously Administered MSC Immune Modulate in Patients with Autoimmunity

Since these initial trials, the use of MSC has been expanded into indications such as heart failure, stroke, Crohn's disease and GVHD. A meta-analysis of over 1000 patients treated with MSC intravenously concluded there was no association between MSC administration and acute infusional toxicity, organ system complications, infection, death or malignancy [129].

In the field of autoimmunity, several clinical trials have been conducted supporting the immune modulatory activity of MSC, which will be described below. In the case of multiple sclerosis, a study of 10 patients with progressive, treatment refractory disease was reported in which patients received autologous MSC intrathecally [130]. The follow-up period was 13-26 months. The EDSS of one patient improved from 5 to 2.5 score. Four patients showed no change in EDSS. Five patients' EDSS increased from 0.5 to 2.5. In the functional system assessment, six patients showed some degree of improvement in their sensory, pyramidal, and cerebellar functions. One showed no difference in clinical assessment and three deteriorated. The result of MRI assessment after 12 months was as following: seven patients with no difference, two showed an extra plaque, and one patient showed decrease in the number of plaques. No patients had adverse events associated with the treatment. This study supported the feasibility and potential of efficacy of MSC therapy.

A similar study was reported in 10 multiple sclerosis patients with progressive disease who were administered autologous MSC intrathecally. Assessment at 3-6 months revealed Expanded Disability Scale Score (EDSS) improvement in 5/7, stabilization in 1/7, and worsening in 1/7 patients. MRI at 3 months revealed new or enlarging lesions in 5/7 and Gadolinium (Gd+) enhancing lesions in 3/7 patients. Vision and low contrast sensitivity testing at 3 months showed improvement in 5/6 and worsening in 1/6 patients [131]. Similar marginal improvement with safety of administration was reported in another intrathecal administration study of 22 patients [132]. Karussis et al also confirmed safety and signals of efficacy in 15 drug resistant patients with secondary progressive MS who received autologous expanded BM-MSC intrathecally. They observed a mean EDSS score improvement from 6.7 (1.0) to 5.9 (1.6). Magnetic resonance imaging visualized the MSCs in the occipital horns of the ventricles, indicating the possible migration in the meninges, subarachnoid space, and spinal cord. Immunological analysis revealed an increase in the proportion of CD4(+)CD25(+) regulatory T cells, a decrease in the proliferative responses of lymphocytes, and the expression of CD40(+), CD83(+), CD86(+), and HLA-DR on myeloid dendritic cells at 24 hours after MSC transplantation [133]. These results are offer the tantalizing suggestion that animal studies demonstrating MSC induction of Treg cells [66, 134-140], may be transferable into the clinical situation. This is further supported by a clinical study of Mohajeri et al which demonstrated MSC treated patients have an increased number of peripheral blood mononuclear cells expressing the Treg marker FoxP3 six months subsequent to treatment [141]. Therapeutic signals in multiple sclerosis were seen not only by intrathecal administration but also by intravenous administration. Connick et al evaluated ten patients with secondary progressive multiple sclerosis administered a mean dose of 1.6×10(6) cells per kg bodyweight (range 1·1-2-0). One patient developed a transient rash shortly after treatment; two patients had self-limiting bacterial infections 3-4 weeks after treatment. No serious adverse events were observed and improvement was noted after treatment in visual acuity and visual evoked response latency, with an increase in optic nerve area [142].

Crohn's disease is characterized by immunologically mediated damage to the gastrointestinal tract and has previously been shown to be responsive to immunologically-active biologicals such as TNF-alpha blockers. A study of 10 adult patients with refractory Crohn's disease (eight females and two males) was reported who underwent bone marrow aspiration under local anaesthesia. Bone marrow MSCs were isolated and expanded ex vivo. Nine patients received two doses of 1-2×10(6) cells/kg body weight, intravenously, 7 days apart. During follow-up, possible side effects and changes in patients' Crohn's disease activity index (CDAI) scores were monitored. Colonoscopies were performed at weeks 0 and 6, and mucosal inflammation was assessed by using the Crohn's disease endoscopic index of severity. MSCs significantly reduced peripheral blood mononuclear cell proliferation in vitro. MSC infusion was without side effects. Baseline median CDAI was 326 (224-378). Three patients showed clinical response (CDAI decrease >70 from baseline) 6 weeks post-treatment; conversely three patients required surgery due to disease worsening [143]. A subsequent study also reported signals of efficacy in which 16 patients (21-55 y old; 6 men) with infliximab- or adalimumab-refractory, endoscopically confirmed, active luminal CD (CD activity index [CDAI], >250). Subjects were given intravenous infusions of allogeneic MSCs (2×10⁶ cells/kg body weight) weekly for 4 weeks. The primary end point was clinical response (decrease in CDAI>100 points) 42 days after the first MSC administration; secondary end points were clinical remission (CDAI, <150), endoscopic improvement (a CD endoscopic index of severity [CDEIS] value, <3 or a decrease by >5), quality of life, level of C-reactive protein, and safety. Among the 15 patients who completed the study, the mean CDAI score was reduced from 370 (median, 327; range, 256-603) to 203 (median, 129) at day 42 (P<0.0001). The mean CDAI scores decreased after each MSC infusion (370 before administration, 269 on day 7, 240 on day 14, 209 on day 21, 182 on day 28, and 203 on day 42). Twelve patients had a clinical response (80%; 95% confidence interval, 72%-88%; mean reduction in CDAI, 211; range 102-367), 8 had clinical remission (53%; range, 43%-64%; mean CDAI at day 42, 94; range, 44-130). Seven patients had endoscopic improvement (47%), for whom the mean CDEIS scores decreased from 21.5 (range, 3.3-33) to 11.0 (range, 0.3-18.5). One patient had a serious adverse event (2 dysplasia-associated lesions), but this probably was not caused by MSCs according to the publication [144].

In the autoimmune condition systemic lupus erythromatosis (SLE), Sun et al reported 4 patients treated with allogeneic BM-MSC. Four cyclophosphamide/glucocorticoid treatment-refractory SLE patients using allogenic MSCT and showed a stable 12-18 months disease remission in all treated patients. The patients benefited an amelioration of disease activity, improvement in serologic markers including decrease in complement C3 and antinuclear antibodies, as well as renal function [145]. These results were reproduced in a larger 58 patient study where patients with SLE were treated either with donor BM-MSC (30 patients) at a million cells/kg dose or a “double transplant” (28 patients) who received a dose of 1 million cells/kg, followed after 7 days of a second dose of 1 million cells/kg. There was a therapeutic benefit in terms of decreased disease scores and serum markers such as anti-nuclear antibodies and creatinine, however there was no additional benefit in the patients that received one dose versus the double dose [146]. A larger study reported 87 patients with persistently active SLE who were refractory to standard treatment or had life-threatening visceral involvement were enrolled. Allogeneic bone marrow or umbilical cord derived MSCs were harvested and infused intravenously (1×10⁶ cells/kg of body weight). Primary outcomes were rates of survival, disease remission and relapse, as well as transplantation related adverse events. Secondary outcomes included SLE disease activity index (SLEDAI) and serologic features. During the 4 years follow up and with a mean follow up period of 27 months, the overall rate of survival was 94% (82/87). Complete clinical remission rate was 28% at 1 year (23/83), 31% at 2 years (12/39), 42% at 3 years (5/12) and 50% at 4 years (3/6). Rates of relapse were 12% (10/83) at 1 year, 18% (7/39) at 2 years, 17% (2/12) at 3 years and 17% (1/6) at 4 years. The overall rate of relapse was 23% (20/87). Disease activity declined as revealed by significant changes in SLEDAI score, levels of serum autoantibodies, albuminand complements. A total of 5 patients (6%) died after MSCT from non-treatment-related events in 4 years follow up, and no transplantation-related adverse event was observed.

In the area of rheumatic diseases, a study in 20 patients with ankylosing spondylitis who were refractory to NSAIDS examined the effect of 4 intravenous infusions (IVI) of MSCs on days 0,7,14, and 21. The percentage of ASAS20 responders (the primary endpoint) at the 4th week and the mean ASAS20 response duration (the secondary endpoint) were used to assess treatment response to MSC infusion and duration of the therapeutic effects. Ankylosing Spondylitis Disease Activity Score Containing C-reactive Protein (ASDAS-CRP) and other pre-established evaluation indices were also adopted to evaluate the clinical effects. Magnetic resonance imaging (MRI) was performed to detect changes of bone marrow edema in the spine. The safety of this treatment was also evaluated. Thirty-one patients were included, and the percentage of ASAS20 responders reached 77.4% at the 4th week and the mean ASAS20 response duration was 7.1 weeks. The mean ASDAS-CRP score decreased from 3.6±0.6 to 2.4±0.5 at the 4th week, and then increased to 3.2±0.8 at the 20th week. The average total inflammation extent (TIE) detected by MRI decreased from 533,482.5 at baseline to 480,692.3 at the 4th week (p>0.05) and 400,547.2 at the 20th week (p<0.05). While the definition of AS may not be a strictly autoimmune rheumatological condition, rheumatoid arthritis (RA) is classically described as an autoimmune condition. The use of umbilical cord MSC in the treatment of RA has been reported in a large clinical trial comprising of 172 patients with active RA who had inadequate responses to traditional medication were enrolled. Patients were divided into two groups for different treatment: disease-modifying anti-rheumatic drugs (DMARDs) plus medium without UC-MSCs, or DMARDs plus UC-MSCs group (4×10⁷ cells per time) via intravenous injection. Adverse events and the clinical information were recorded. No serious adverse effects were observed during or after infusion. The serum levels of tumor necrosis factor-alpha and interleukin-6 decreased after the first UC-MSCs treatment (P<0.05). The percentage of CD4⁺CD25⁺Foxp3⁺ regulatory T cells of peripheral blood was increased (P<0.05). The treatment induced a significant remission of disease according to the American College of Rheumatology improvement criteria, the 28-joint disease activity score, and the Health Assessment Questionnaire. The therapeutic effects maintained for 3-6 months without continuous administration, correlating with the increased percentage of regulatory T cells of peripheral blood. Repeated infusion after this period can enhance the therapeutic efficacy. In comparison, there were no such benefits observed in control group of DMARDS plus medium without UC-MSCs.

Allograft rejection is a classical immunological phenomena, with the effective treatment of acute allograft rejection by immune suppressants such as cyclosporine having made possible the field of organ transplantation. Unfortunately, the lifelong use of immune suppressants, causes numerous adverse events, including nephrotoxicity and susceptibility to opportunistic infections [147]. Additionally, current immune suppressants do not address the issue of chronic organ rejection, which is the major cause of graft failure [148]. Although induction of donor specific tolerance (eg recipients being able to be taken off immune suppression) has not been achieved on a routine basis, in a 4 patient report where tolerance was achieved, an association with generation of Treg cells was reported [149]. Several studies have assessed the use of MSC as immune suppressants. Perico et al utilized autologous BM-MSC (1.7 and 2 million cells/kg, respectively) infusion in two recipients of kidneys from living-related donors. Patients were given T cell-depleting induction therapy and maintenance immunosuppression with cyclosporine and mycophenolate mofetil. On day 7 posttransplant, MSCs were administered intravenously. Clinical and immunomonitoring of MSC-treated patients was performed up to day 360 postsurgery. Serum creatinine levels increased 7 to 14 days after cell infusion in both MSC-treated patients. One year post-transplant, both MSC-treated patients are in good health with stable graft function. A progressive increase of the percentage of CD4+CD25highFoxP3+CD127− Treg and a marked inhibition of memory CD45RO+RA-CD8+ T cell expansion were observed posttransplant. Patient T cells showed a profound reduction of CD8+ T cell activity [150]. A subsequent 6 patient study examined effects of 1 million autologous BM-MSC/kg in recipients of allogeneic kidney grafts. five of the six patients displayed a donor-specific downregulation of the peripheral blood mononuclear cell proliferation assay, not reported in patients without MSC treatment. Autologous BM MSC treatment in transplant recipients with subclinical rejection and interstitial fibrosis/tubular atrophy is clinically feasible and safe, and the findings are suggestive of systemic immunosuppression by MSC [151]. In a larger study, Tan et al reported on 159 patients enrolled in this single-site, prospective, open-label, randomized study in which patients were inoculated with marrow-derived autologous MSC (1-2×10(6)/kg) at kidney reperfusion and two weeks later. Fifty-three patients received standard-dose and 52 patients received low-dose calcineurin inhibitors (CNIs) (80% of standard); 51 patients in the control group received anti-IL-2 receptor antibody plus standard-dose calcineurin inhibitors. The authors reported patient and graft survival at 13 to 30 months to be similar in all groups. After 6 months, 4 of 53 patients (7.5%) in the autologous MSC plus standard-dose CNI group (95% CI, 0.4%-14.7%; P=0.04) and 4 of 52 patients (7.7%) in the low-dose group (95% CI, 0.5%-14.9%; P=0.046) compared with 11 of 51 controls (21.6%; 95% CI, 10.5%-32.6%) had biopsy-confirmed acute rejection. None of the patients in either autologous MSC group had glucorticoid-resistant rejection, whereas 4 patients (7.8%) in the control group did (95% CI, 0.6%-15.1%; overall P=0.02). Renal function recovered faster among both MSC groups showing increased eGFR levels during the first month after surgery than the control group. Patients receiving standard-dose CNI had a mean difference of 6.2 mL/min per 1.73 m(2) (95% CI, 0.4-11.9; P=.04) and those in the low-dose CNI of 10.0 mL/min per 1.73 m(2) (95% CI, 3.8-16.2; P=0.002). Also, during the 1-year follow-up, combined analysis of MSC-treated groups revealed significantly decreased risk of opportunistic infections than the control group (hazard ratio, 0.42; 95% CI, 0.20-0.85, P=0.02) [152].

Thus it appears that MSC possess in vitro ability to immune modulate, which was demonstrated in numerous animal models. Mechanistically, this may be working through several means, including stimulation of Treg cells. Data in clinical trials in multiple sclerosis, Crohn's disease, rheumatoid arthritis and transplant rejection support the notion that MSC are capable of inducing Tregs and in some situations exerting a therapeutic benefit, however studies larger scale studies are needed.

### Mechanisms of Immune Modulation by MSC

MSC modulate the immune system at several levels, we will discuss below the effects of MSC on various aspects of immune response induction from antigen presentation to effector function. Dendritic cells (DC) are considered the primary sentinels of the immune response, playing a key role in determining whether productive immunity will ensure, versus stimulation of T regulatory cells and suppression of immunity [153, 154]. Although various subtypes of DC exist, with varying specialized functions, one of the common themes appears to be that immature myeloid type DC reside in an immature state in the periphery, which engulf antigens and present in a tolerogenic manner to T cells in the lymph nodes. This is one of the mechanisms by which self tolerance is maintained. Specifically, although small numbers of autoreactive T cells escape the thymic selection process, these T cells are either anergized, or their activity suppressed by T regulatory cells generated as a result of immature dendritic cells presenting self antigens to autoreactive T cells. In contrast, in the presence of “danger” signals, such as toll like receptor agonists, immature DC take a mature phenotype, characterized by high expression of costimulatory molecules, and subsequently induce T cell activation [155-157]. In the context of T1D it has previously been demonstrated that targeting of diabetogenic autoantigens to immature DC leads to prevention of disease [158]. Administration of immature DC into 10 T1D patients resulted in increased C-peptide levels with some evidence of immunomodulatory activity[159].

Given the fundamental role of the DC in controlling immunity versus tolerance, the manipulation of DC maturation by MSC would strongly support an immune modulatory role of MSC. Early studies suggested that MSC may inhibit the ability of DC to stimulate CD4 and CD8 cells using in vitro systems, however, it was demonstrated that MSC also inhibited T cell activation directly [160]. Subsequently, Zhang et al performed a definitive study in which bone marrow MSC were cultured directly with monocytes which were stimulated to differentiate into DC using a standard IL-4 and GM-CSF protocol in the murine system. It was found that MSCs inhibit the up-regulation of CD1a, CD40, CD80, CD86, and HLA-DR during DC differentiation and prevent an increase of CD40, CD86, and CD83 expression during DC maturation. MSCs supernatants had no effect on DCs differentiation, but they inhibited the up-regulation of CD83 during maturation. Both MSCs and their supernatants interfered with endocytosis of DCs, decreased their capacity to secret IL-12 and activate alloreactive T cells [161]. Using the human system, Aggarwal et al cultured Prochymal BM derived MSC together with DC that were polarized to generate Th1 promoting cytokines (DC1) and DC polarized to generate Th2 cytokines (DC2). MSC were demonstrated to inhibit production of TNF-alpha and IL-12 in DC1 cells while increase production of IL-10 in DC2 cells [161]. The concept of MSC inducing immaturity in DC was further demonstrated in mixed lymphocyte reactions where it was shown that addition of MSC would suppress MLR only in the presence of antigen presenting cells [162]. Interestingly, the addition of DC maturation agents such as LPS or antiCD40 antibody was capable of overcoming MSC mediated suppression, thus implying that inhibition of DC immunogenic activities by MSC is a reversible process. The general ability of MSC to suppress DC immune stimulatory activities, through inhibition of maturation and costimulatory molecule expression was replicated in other studies [163-168]. Other methods by which MSC inhibit DC activity include blocking the physical interaction with T cells [163], blocking DC progenitor entry into cell cycle [163], production of TSG-6 [169], induction of Notch in DC progenitors [170], and arresting DC migration into lymph nodes in vivo [171-173].

Some of the immune suppressive effects of MSC appear to be inducible by the presence of local inflammation. For example, a recent study showed that TLR activation on MSC increases ability of the MSC to suppress T cell activation through blockade of DC maturation [174]. Other studies have shown that treatment of MSC with inflammatory mediators such as IL-1 beta actually stimulates production of cytokines such as IL-10 that block DC maturation. IL-1 treated MSC possess superior in vivo ability to suppress inflammatory diseases such as DSS induced colitis [175]. Similar augmentation of anti-inflammatory activity of MSC by pretreatment with inflammatory cytokines was also reported by treatment with IFN-gamma [176-178]. On a cellular level it has been reported that coculture of MSC with monocytes leads to enhanced immune suppressive activities of the MSC, in part through monocyte produced IL-1 [179].

Inhibition of T cell reactivity by MSC has been widely described. One of the initial publications supporting this assessed baboon MSCs in vitro for their ability to elicit a proliferative response from allogeneic lymphocytes, to inhibit an ongoing allogeneic response, and to inhibit a proliferative response to potent T-cell mitogens. It was found that the MSCs failed to elicit a proliferative response from allogeneic lymphocytes. MSCs added into a mixed lymphocyte reaction, either on day 0 or on day 3, or to mitogen-stimulated lymphocytes, led to a greater than 50% reduction in proliferative activity. This effect could be maximized by escalating the dose of MSCs and could be reduced with the addition of exogenous IL-2. In vivo administration of MSCs led to prolonged skin graft survival when compared to control animals [180, 181]. Inhibition of T cell proliferation could not be restored by costimulation or pretreatment of the MSC with IFN-gamma [182], which is intriguing given that the previous study mentioned showed IL-2 could overcome MSC mediated suppression. In vivo studies using humanized mice demonstrated that human MSC were capable of suppressing human T cell responses in vivo, both allogenic and antigen-specific responses [183]. Inhibition of T cell activity seems to be not limited to proliferation but also was demonstrated to include suppression of cytotoxic activity of CD8 T cells [184, 185].

Several mechanisms have been reported for MSC suppression of T cell activation including inhibition of IL-2 receptor alpha (CD25) [186], induction of division arrest [187, 188], induction of T cell anergy directly [189] or via immature DC [168], stimulation of apoptosis of activated T cells [190, 191], blockade of IL-2 signaling and induction of PGE2 production [192-197], induction of TGF-beta[198], production of HLA-G [199], expression of serine protease inhibitor 6 [200], stimulation of nitric oxide release [201-203], stimulation of indolamine 2,3 deoxygenase [204-207], expression of adenosine generating ectoenzymes such as CD39 and CD73 [208-210], Galectin expression[211, 212], induction of hemoxygenase 1[213, 214], activation of the PD1 pathway [211, 215-217], Fas ligand expression [67, 218], CD200 expression [219], Th2 deviation [74, 220, 221], inhibition of Th17 differentiation [222-226], TSG-6 expression [227], NOTCH-1 expression [228], stimulation of Treg cell generation [66, 134-140]. Mechanisms of Treg generation may be direct, or may be through modulation of DC. It has been reported by us and others, that activation of T cells in the absence of costimulatory signals leads to generation of immune suppressive CD4+ CD25+ T regulatory (Treg) cells [229, 230]. Thus local activation of immunity in lymph nodes would theoretically be associated with reduced costimulatory molecule expression DC after MSC administration, which may predispose to Treg generation. Conversely, it is known that Tregs are involved in maintaining DC in the DC2 phenotype [231]. Indeed numerous studies have demonstrated the ability of MSC to induce Treg cells [136, 137, 139, 232]

T cell suppressive properties of MSC have not been restricted to the bone marrow MSC types and have been reported in MSC derived from a wide variety of tissues including tooth [177], placental matrix [233, 234], adipose tissue [78, 235-239], cord blood [240], muscle [241], amnion [199, 242], and Wharton's Jelly [243, 244].

In addition to modulation of B cell activity, MSC have been demonstrated to suppress various aspects of B cell activation. Corcione et al [245], used human MSCs isolated from the bone marrow and B cells purified from the peripheral blood of healthy donors in a coculture with different B-cell tropic stimuli. B-cell proliferation was inhibited by MSCs through an arrest in the G0/G1 phase of the cell cycle and not through the induction of apoptosis. A major mechanism of B-cell suppression was MSC production of soluble factors, as indicated by transwell experiments. MSCs inhibited B-cell differentiation as indicated by the observation that IgM, IgG, and IgA production was significantly impaired. CXCR4, CXCR5, and CCR7 B-cell expression, as well as chemotaxis to CXCL12, the CXCR4 ligand, and CXCL13, the CXCR5 ligand, were significantly down-regulated by MSCs, suggesting that these cells affect chemotactic properties of B cells, which was similarly reported for MSC suppressive activities on DC [171-173]. Similar inhibition of B cell function was observed in a study assessing human B cells generating alloreactive antibodies, a dose dependent inhibition of antibody production was observed in coculture with MSC [246]. Mechanistically, it has been reported that B cells are inhibited by MSC through suppression of cell cycle entry, as well as inhibition of DC induced B cell maturation [247]. The suppression of B cell differentiation has been reported to be dependent on MSC production of the chemokine CCL2, which in turn inhibits STAT3 in the B cell [248], this has also been associated with suppression of BLIMP-1 production [249, 250]. MSC inhibition of B cell activation was also observed in vivo in a murine model of systemic lupus erythramatosis [251]. As in the situation of MSC inhibition of T cell activation, IFN-gamma pretreatment of MSC upregulates ability to suppress B cell function [252]. Another similarity between MSC mediated suppression of T cells and suppression of B cells is that MSC appear to induce generation of a “regulatory B cell” subset that has activity in vivo against autoimmune conditions [253]. Suppression of antibody production was observed not only with BM-MSC but also with adipose derived MSC [254, 255], umbilical cord MSC [256] and periodontal ligament [257].

### REFERENCES

1. Ramsdell, F. and B. J. Fowlkes, Clonal deletion versus clonal anergy: the role of the thymus in inducing self tolerance. Science, 1990. 248(4961): p. 1342-8.

2. Apostolou, I., et al., Origin of regulatory T cells with known specificity for antigen. Nat Immunol, 2002. 3(8): p. 756-63.

3. Aschenbrenner, K., et al., Selection of Foxp3+ regulatory T cells specific for self antigen expressed and presented by Aire+ medullary thymic epithelial cells. Nat Immunol, 2007. 8(4): p. 351-8.

4. Vacchio, M. S. and R. J. Hodes, Fetal expression of Fas ligand is necessary and sufficient for induction of CD8 T cell tolerance to the fetal antigen H-Y during pregnancy. J Immunol, 2005. 174(8): p. 4657-61.

5. D'Addio, F., et al., The link between the PDL1 costimulatory pathway and Th17 in fetomaternal tolerance. J Immunol, 2011. 187(9): p. 4530-41.

6. Kuroki, K. and K. Maenaka, Immune modulation of HLA-G dimer in maternal-fetal interface. Eur J Immunol, 2007. 37(7): p. 1727-9.

7. Erlebacher, A., et al., Constraints in antigen presentation severely restrict T cell recognition of the allogeneic fetus. J Clin Invest, 2007. 117(5): p. 1399-411.

8. Chen, T., et al., Self-specific memory regulatory T cells protect embryos at implantation in mice. J Immunol, 2013. 191(5): p. 2273-81.

9. Harimoto, H., et al., Inactivation of tumor-specific CD8(+) CTLs by tumor-infiltrating tolerogenic dendritic cells. Immunol Cell Biol, 2013. 91(9): p. 545-55.

10. Ney, J. T., et al., Autochthonous liver tumors induce systemic T cell tolerance associated with T cell receptor down-modulation. Hepatology, 2009. 49(2): p. 471-81.

11. Cheung, A. F., et al., Regulated expression of a tumor-associated antigen reveals multiple levels of T-cell tolerance in a mouse model of lung cancer. Cancer Res, 2008. 68(22): p. 9459-68.

12. Bai, A., et al., Rapid tolerization of virus-activated tumor-specific CD8+ T cells in prostate tumors of TRAMP mice. Proc Natl Acad Sci U S A, 2008. 105(35): p. 13003-8.

13. Jacobs, J. F., et al., Regulatory T cells in melanoma: the final hurdle towards effective immunotherapy? Lancet Oncol, 2012. 13(1): p. e32-42.

14. Pedroza-Gonzalez, A., et al., Activated tumor-infiltrating CD4+ regulatory T cells restrain antitumor immunity in patients with primary or metastatic liver cancer. Hepatology, 2013. 57(1): p. 183-94.

15. Donkor, M. K., et al., T cell surveillance of oncogene-induced prostate cancer is impeded by T cell-derived TGF-beta1 cytokine. Immunity, 2011. 35(1): p. 123-34.

16. Whiteside, T. L., Down-regulation of zeta-chain expression in T cells: a biomarker of prognosis in cancer? Cancer Immunol Immunother, 2004. 53(10): p. 865-78.

17. Whiteside, T. L., Signaling defects in T lymphocytes of patients with malignancy. Cancer Immunol Immunother, 1999. 48(7): p. 346-52.

18. Reichert, T. E., et al., Signaling abnormalities, apoptosis, and reduced proliferation of circulating and tumor-infiltrating lymphocytes in patients with oral carcinoma. Clin Cancer Res, 2002. 8(10): p. 3137-45.

19. Faria, A. M. and HL. Weiner, Oral tolerance. Immunol Rev, 2005. 206: p. 232-59.

20. Weiner, H. L., Induction and mechanism of action of transforming growth factor-beta-secreting Th3 regulatory cells. Immunol Rev, 2001. 182: p. 207-14.

21. Fukaura, H., et al., Induction of circulating myelin basic protein and proteolipid protein-specific transforming growth factor-beta1-secreting Th3T cells by oral administration of myelin in multiple sclerosis patients. J Clin Invest, 1996. 98(1): p. 70-7.

22. Palomares, O., et al., Induction and maintenance of allergen-specific FOXP3+ Treg cells in human tonsils as potential first-line organs of oral tolerance. J Allergy Clin Immunol, 2012. 129(2): p. 510-20, 520 e1-9.

23. Yamashita, H., et al., Overcoming food allergy through acquired tolerance conferred by transfer of Tregs in a murine model. Allergy, 2012. 67(2): p. 201-9.

24. Park, K. S., et al., Type II collagen oral tolerance; mechanism and role in collagen-induced arthritis and rheumatoid arthritis. Mod Rheumatol, 2009.

25. Womer, K. L., et al., A pilot study on the immunological effects of oral administration of donor major histocompatibility complex class II peptides in renal transplant recipients. Clin Transplant, 2008. 22(6): p. 754-9.

26. Faria, A. M. and H. L. Weiner, Oral tolerance: therapeutic implications for autoimmune diseases. Clin Dev Immunol, 2006. 13(2-4): p. 143-57.

27. Park, M. J., et al., A distinct tolerogenic subset of splenic IDO(+)CD11b(+) dendritic cells from orally tolerized mice is responsible for induction of systemic immune tolerance and suppression of collagen-induced arthritis. Cell Immunol, 2012. 278(1-2): p. 45-54.

28. Thompson, H. S., et al., Suppression of collagen induced arthritis by oral administration of type II collagen: changes in immune and arthritic responses mediated by active peripheral suppression. Autoimmunity, 1993. 16(3): p. 189-99.

29. Song, F., et al., The thymus plays a role in oral tolerance induction in experimental autoimmune encephalomyelitis. Ann N Y Acad Sci, 2004. 1029: p. 402-4.

30. Hanninen, A. and L. C. Harrison, Mucosal tolerance to prevent type 1 diabetes: can the outcome be improved in humans? Rev Diabet Stud, 2004. 1(3): p. 113-21.

31. Wei, W., et al., A multicenter, double-blind, randomized, controlled phase III clinical trial of chicken type II collagen in rheumatoid arthritis. Arthritis Res Ther, 2009. 11(6): p. R180.

32. Thurau, S. R., et al., Molecular mimicry as a therapeutic approach for an autoimmune disease: oral treatment of uveitis patients with an MHC-peptide crossreactive with autoantigen—first results. Immunol Lett, 1997. 57(1-3): p. 193-201.

33. Weiner, H. L., et al., Double-blind pilot trial of oral tolerization with myelin antigens in multiple sclerosis. Science, 1993. 259(5099): p. 1321-4.

34. Prockop, D. J., Marrow stromal cells as stem cells for nonhematopoietic tissues. Science, 1997. 276(5309): p. 71-4.

35. Friedenstein, A. J., et al., Heterotopic of bone marrow.Analysis of precursor cells for osteogenic and hematopoietic tissues. Transplantation, 1968. 6(2): p. 230-47.

36. Zannettino, A. C., et al., Multipotential human adipose-derived stromal stem cells exhibit a perivascular phenotype in vitro and in vivo. J Cell Physiol, 2008. 214(2): p. 413-21.

37. Hoogduijn, M. J., et al., Human heart, spleen, and perirenal fat-derived mesenchymal stem cells have immunomodulatory capacities. Stem Cells Dev, 2007. 16(4): p. 597-604.

38. Chao, K. C., et al., Islet-like clusters derived from mesenchymal stem cells in Wharton's Jelly of the human umbilical cord for transplantation to control type 1 diabetes. PLoS ONE, 2008. 3(1): p. e1451.

39. Jo, Y. Y., et al., Isolation and characterization of postnatal stem cells from human dental tissues. Tissue Eng, 2007. 13(4): p. 767-73.

40. He, Q., C. Wan, and G. Li, Concise review: multipotent mesenchymal stromal cells in blood. Stem Cells, 2007. 25(1): p. 69-77.

41. Oh, W., et al., Immunological properties of umbilical cord blood-derived mesenchymal stromal cells. Cell Immunol, 2008.

42. Meng, X., et al., Endometrial regenerative cells: a novel stem cell population. J Transl Med, 2007. 5: p. 57.

43. Hida, N., et al., Novel Cardiac Precursor-Like Cells from Human Menstrual Blood-Derived Mesenchymal Cells. Stem Cells, 2008.

44. Patel, A. N., et al., Multipotent menstrual blood stromal stem cells: isolation, characterization, and differentiation. Cell Transplant, 2008. 17(3): p. 303-11.

45. Pittenger, M. F. and B. J. Martin, Mesenchymal stem cells and their potential as cardiac therapeutics. Circ Res, 2004. 95(1): p. 9-20.

46. Sugiyama, T., et al., Maintenance of the hematopoietic stem cell pool by CXCL12-CXCR4 chemokine signaling in bone marrow stromal cell niches. Immunity, 2006. 25(6): p. 977-88.

47. Anthony, B. A. and D. C. Link, Regulation of hematopoietic stem cells by bone marrow stromal cells. Trends Immunol, 2013.

48. Greenbaum, A., et al., CXCL12 in early mesenchymal progenitors is required for haematopoietic stem-cell maintenance. Nature, 2013. 495(7440): p. 227-30.

49. Lazarus, H. M., et al., Ex vivo expansion and subsequent infusion of human bone marrow-derived stromal progenitor cells (mesenchymal progenitor cells): implications for therapeutic use. Bone marrow transplantation, 1995. 16(4): p. 557-64.

50. Koc, O. N., et al., Rapid hematopoietic recovery after coinfusion of autologous-blood stem cells and culture-expanded marrow mesenchymal stem cells in advanced breast cancer patients receiving high-dose chemotherapy. Journal of clinical oncology : official journal of the American Society of Clinical Oncology, 2000. 18(2): p. 307-16.

51. Nassiri, S. M. and R. Rahbarghazi, Interactions of mesenchymal stem cells with endothelial cells. Stem Cells Dev, 2013.

52. Duffy, G. P. and C. C. Herron, Mesenchymal stem cells to augment therapeutic angiogenesis in hind-limb ischemia models: how important is their source? Stem Cell Res Ther, 2013. 4(5): p. 131.

53. Watt, S. M., et al., The angiogenic properties of mesenchymal stem/stromal cells and their therapeutic potential. Br Med Bull, 2013.

54. Carrion, B., et al., Bone marrow-derived mesenchymal stem cells enhance angiogenesis via their alpha6betal integrin receptor. Exp Cell Res, 2013. 319(19): p. 2964-76.

55. Kong, P., et al., Placenta mesenchymal stem cell accelerates wound healing by enhancing angiogenesis in diabetic Goto-Kakizaki (GK) rats. Biochem Biophys Res Commun, 2013. 438(2): p. 410-9.

56. Cunha, F. F., et al., A comparison of the reparative and angiogenic properties of mesenchymal stem cells derived from the bone marrow of BALB/c and C57/BL6 mice in a model of limb ischemia. Stem Cell Res Ther, 2013. 4(4): p. 86.

57. Jin, H., P. R. Sanberg, and R. J. Henning, Human umbilical cord blood mononuclear cell-conditioned media inhibits hypoxic-induced apoptosis in human coronary artery endothelial cells and cardiac myocytes by activation of the survival protein Akt. Cell Transplant, 2013. 22(9): p. 1637-50.

58. Doom, J., et al., Therapeutic applications of mesenchymal stromal cells: paracrine effects and potential improvements. Tissue Eng Part B Rev, 2012. 18(2): p. 101-15.

59. Cassatella, M. A., et al., Toll-like receptor-3-activated human mesenchymal stromal cells significantly prolong the survival and function of neutrophils. Stem Cells, 2011. 29(6): p. 1001-11.

60. Karaoz, E., et al., Protection of rat pancreatic islet function and viability by coculture with rat bone marrow-derived mesenchymal stem cells. Cell Death Dis, 2010. 1: p. e36.

61. Hauser, P. V., et al., Stem cells derived from human amniotic fluid contribute to acute kidney injury recovery. Am J Pathol, 2010. 177(4): p. 2011-21.

62. Bartosh, T. J., et al., Aggregation of human mesenchymal stromal cells (MSCs) into 3D spheroids enhances their antiinflammatory properties. Proc Natl Acad Sci U S A, 2010. 107(31): p. 13724-9.

63. He, A., et al., The antiapoptotic effect of mesenchymal stem cell transplantation on ischemic myocardium is enhanced by anoxic preconditioning. Can J Cardiol, 2009. 25(6): p. 353-8.

64. Chen, Q. Q., et al., Mesenchymal stem cells alleviate TNBS-induced colitis by modulating inflammatory and autoimmune responses. World J Gastroenterol, 2013. 19(29): p. 4702-17.

65. Abumaree, M. H., et al., Human placental mesenchymal stem cells (pMSCs) play a role as immune suppressive cells by shifting macrophage differentiation from inflammatory M1 to anti-inflammatory M2 macrophages. Stem Cell Rev, 2013. 9(5): p. 620-41.

66. Melief, S. M., et al., Multipotent stromal cells induce human regulatory T cells through a novel pathway involving skewing of monocytes toward anti-inflammatory macrophages. Stem Cells, 2013. 31(9): p. 1980-91.

67. Gu, Y. Z., et al., Different roles of PD-L and FasL in immunomodulation mediated by human placenta-derived mesenchymal stem cells. Hum Immunol, 2013. 74(3): p. 267-76.

68. Hof-Nahor, I., et al., Human mesenchymal stem cells shift CD8+ T cells towards a suppressive phenotype by inducing tolerogenic monocytes. J Cell Sci, 2012. 125(Pt 19): p. 4640-50.

69. Karlsson, H., et al., Stromal cells from term fetal membrane are highly suppressive in allogeneic settings in vitro. Clin Exp Immunol, 2012. 167(3): p. 543-55.

70. Abumaree, M., et al., Immunosuppressive properties of mesenchymal stem cells. Stem Cell Rev, 2012. 8(2): p. 375-92.

71. Li, Y., et al., Bone marrow mesenchymal stem cells reduce the antitumor activity of cytokine-induced killer/natural killer cells in K562 NOD/SCID mice. Ann Hematol, 2011. 90(8): p. 873-85.

72. Zhou, Y., et al., The therapeutic efficacy of human adipose tissue-derived mesenchymal stem cells on experimental autoimmune hearing loss in mice. Immunology, 2011. 133(1): p. 133-40.

73. Kavanagh, H. and B. P. Mahon, Allogeneic mesenchymal stem cells prevent allergic airway inflammation by inducing murine regulatory T cells. Allergy, 2011. 66(4): p. 523-31.

74. Zanone, M. M., et al., Human mesenchymal stem cells modulate cellular immune response to islet antigen glutamic acid decarboxylase in type 1 diabetes. J Clin Endocrinol Metab, 2010. 95(8): p. 3788-97.

75. Rafei, M., et al., Allogeneic mesenchymal stem cells for treatment of experimental autoimmune encephalomyelitis. Mol Ther, 2009. 17(10): p. 1799-803.

76. Ding, Y., et al., Mesenchymal stem cells prevent the rejection of fully allogenic islet grafts by the immunosuppressive activity of matrix metalloproteinase-2 and -9. Diabetes, 2009. 58(8): p. 1797-806.

77. Gonzalez, M. A., et al., Treatment of experimental arthritis by inducing immune tolerance with human adipose-derived mesenchymal stem cells. Arthritis Rheum, 2009. 60(4): p. 1006-19.

78. Gonzalez, M. A., et al., Adipose-derived mesenchymal stem cells alleviate experimental colitis by inhibiting inflammatory and autoimmune responses. Gastroenterology, 2009. 136(3): p. 978-89.

79. Ryan, J. M., et al., Mesenchymal stem cells avoid allogeneic rejection. J Inflamm (Lond), 2005. 2: p. 8.

80. Kim, S. J., et al., Intravenous transplantation of mesenchymal stem cells preconditioned with early phase stroke serum: current evidence and study protocol for a randomized trial. Trials, 2013. 14(1): p. 317.

81. Lee, J. S., et al., A long-term follow-up study of intravenous autologous mesenchymal stem cell transplantation in patients with ischemic stroke. Stem Cells, 2010. 28(6): p. 1099-106.

82. Bang, O. Y., et al., Autologous mesenchymal stem cell transplantation in stroke patients. Ann Neurol, 2005. 57(6): p. 874-82.

83. Bhasin, A., et al., Stem cell therapy: a clinical trial of stroke. Clin Neurol Neurosurg, 2013. 115(7): p. 1003-8.

84. Bartunek, J., et al., Cardiopoietic stem cell therapy in heart failure: the C-CURE (Cardiopoietic stem Cell therapy in heart failURE)multicenter randomized trial with lineage-specified biologics. J Am Coll Cardiol, 2013. 61(23): p. 2329-38.

85. Yang, Z., et al., A novel approach to transplanting bone marrow stem cells to repair human myocardial infarction: delivery via a noninfarct-relative artery. Cardiovasc Ther, 2010. 28(6): p. 380-5.

86. Weiss, D. J., et al., A placebo-controlled, randomized trial of mesenchymal stem cells in COPD. Chest, 2013. 143(6): p. 1590-8.

87. Shi, M., et al., Human mesenchymal stem cell transfusion is safe and improves liver function in acute-on-chronic liver failure patients. Stem Cells Transl Med, 2012. 1(10): p. 725-31.

88. Horwitz, E. M., et al., Isolated allogeneic bone marrow-derived mesenchymal cells engraft and stimulate growth in children with osteogenesis imperfecta: Implications for cell therapy of bone. Proc Natl Acad Sci U S A, 2002. 99(13): p. 8932-7.

89. Koc, O. N., et al., Allogeneic mesenchymal stem cell infusion for treatment of metachromatic leukodystrophy (MLD) and Hurler syndrome (MPS-IH). Bone Marrow Transplant, 2002. 30(4): p. 215-22.

90. Ichim, T. E., et al., Mesenchymal stem cells as anti-inflammatories: implications for treatment of Duchenne muscular dystrophy. Cell Immunol, 2010. 260(2): p. 75-82.

91. Wang, C., et al., Towards whole-body imaging at the single cell level using ultra-sensitive stem cell labeling with oligo-arginine modified upconversion nanoparticles. Biomaterials, 2012. 33(19): p. 4872-81.

92. Ahluwalia, A. and A. S. Tarnawski, Critical role of hypoxia sensor—HIF-1alpha in VEGF gene activation. Implications for angiogenesis and tissue injury healing. Curr Med Chem, 2012. 19(1): p. 90-7.

93. Imtiyaz, H. Z. and M. C. Simon, Hypoxia-inducible factors as essential regulators of inflammation. Curr Top Microbiol Immunol, 2010. 345: p. 105-20.

94. Youn, S. W., et al., COMP-Ang1 stimulates HIF-1 alpha-mediated SDF-1 overexpression and recovers ischemic injury through BM-derived progenitor cell recruitment. Blood, 2011. 117(16): p. 4376-86.

95. Ceradini, D. J., et al., Progenitor cell trafficking is regulated by hypoxic gradients through HIF-1 induction of SDF-1. Nat Med, 2004. 10(8): p. 858-64.

96. Crisostomo, P. R., et al., Human mesenchymal stem cells stimulated by TNF-alpha, LPS, or hypoxia produce growth factors by an NF kappa B-but not JNK-dependent mechanism. Am J Physiol Cell Physiol, 2008. 294(3): p. C675-82.

97. Rasmussen, J. G., et al., Prolonged hypoxic culture and trypsinization increase the pro-angiogenic potential of human adipose tissue-derived stem cells. Cytotherapy, 2011. 13(3): p. 318-28.

98. Yust-Katz, S., et al., Placental mesenchymal stromal cells induced into neurotrophic factor-producing cells protect neuronal cells from hypoxia and oxidative stress. Cytotherapy, 2012. 14(1): p. 45-55.

99. Iida, K., et al., Hypoxia enhances colony formation and proliferation but inhibits differentiation of human dental pulp cells. Arch Oral Biol, 2010. 55(9): p. 648-54.

100. Efimenko, A., et al., Angiogenic properties of aged adipose derived mesenchymal stem cells after hypoxic conditioning. J Transl Med, 2011. 9: p. 10.

101. Chang, C. P., et al., Hypoxic preconditioning enhances the therapeutic potential of the secretome from cultured human mesenchymal stem cells in experimental traumatic brain injury. Clin Sci (Lond), 2013. 124(3): p. 165-76.

102. Yu, J., et al., Hypoxia preconditioned bone marrow mesenchymal stem cells promote liver regeneration in a rat massive hepatectomy model. Stem Cell Res Ther, 2013. 4(4): p. 83.

103. Li, J. H., N. Zhang, and J. A. Wang, Improved anti-apoptotic and anti-remodeling potency of bone marrow mesenchymal stem cells by anoxic pre-conditioning in diabetic cardiomyopathy. J Endocrinol Invest, 2008. 31(2): p. 103-10.

104. Hague, N., et al., Hypoxic culture conditions as a solution for mesenchymal stem cell based regenerative therapy. ScientificWorldJournal, 2013. 2013: p. 632972.

105. Hawkins, K. E., T. V. Sharp, and T. R. McKay, The role of hypoxia in stem cell potency and differentiation. Regen Med, 2013. 8(6): p. 771-82.

106. Berniakovich, I. and M. Giorgio, Low oxygen tension maintains multipotency, whereas normoxia increases differentiation of mouse bone marrow stromal cells. Int J Mol Sci, 2013. 14(1): p. 2119-34.

107. Le Blanc, K., et al., Mesenchymal stem cells for treatment of steroid-resistant, severe, acute graft-versus-host disease: a phase II study. Lancet, 2008. 371(9624): p. 1579-86.

108. Ning, H., et al., The correlation between cotransplantation of mesenchymal stem cells and higher recurrence rate in hematologic malignancy patients: outcome of a pilot clinical study. Leukemia, 2008. 22(3): p. 593-9.

109. Ball, L., et al., Third party mesenchymal stromal cell infusions fail to induce tissue repair despite successful control of severe grade IV acute graft-versus-host disease in a child with juvenile myelo-monocytic leukemia. Leukemia, 2008. 22(6): p. 1256-7.

110. Ringden, O., et al., Mesenchymal stem cells for treatment of therapy-resistant graft-versus-host disease. Transplantation, 2006. 81(10): p. 1390-7.

111. Le Blanc, K., et al., Treatment of severe acute graft-versus-host disease with third party haploidentical mesenchymal stem cells. Lancet, 2004. 363(9419): p. 1439-41.

112. Muller, I., et al., Application of multipotent mesenchymal stromal cells in pediatric patients following allogeneic stem cell transplantation. Blood Cells Mol Dis, 2008. 40(1): p. 25-32.

113. Roemeling-van Rhijn, M., et al., Effects of Hypoxia on the Immunomodulatory Properties of Adipose Tissue-Derived Mesenchymal Stem cells. Front Immunol, 2013. 4: p. 203.

114. English, K., et al., Mesoangioblasts suppress T cell proliferation through IDO and PGE-2-dependent pathways. Stem Cells Dev, 2013. 22(3): p. 512-23.

115. Engela, A. U., et al., Interaction between adipose tissue-dirived mesenchymal stem cells and regulatory T-cells. Cell Transplant, 2013. 22(1): p. 41-54.

116. Jui, H. Y., et al., Autologous mesenchymal stem cells prevent transplant arteriosclerosis by enhancing local expression of interleukin-10, interferon-gamma, and indoleamine 2,3-dioxygenase. Cell Transplant, 2012. 21(5): p. 971-84.

117. Huang, W. H., et al., Hypoxic mesenchymal stem cells engraft and ameliorate limb ischaemia in allogeneic recipients. Cardiovasc Res, 2013.

118. Skurkovich, B. and S. Skurkovich, Anti-interferon-gamma antibodies in the treatment of autoimmune diseases. Curr Opin Mol Ther, 2003. 5(1): p. 52-7.

119. Rong, L. J., et al., [Effects of interferon-gamma on biological characteristics and immunomodulatory property of human umbilical cord-derived mesenchymal stem cells]. Zhongguo Shi Yan Xue Ye Xue Za Zhi, 2012. 20(2): p. 421-6.

120. Kang, J. W., et al., Immunomodulatory effects of human amniotic membrane-derived mesenchymal stem cell. J Vet Sci, 2012. 13(1): p. 23-31.

121. Lin, W., et al., Activated T cells modulate immunosuppression by embryonic-and bone marrow-derived mesenchymal stromal cells through a feedback mechanism. Cytotherapy, 2012. 14(3): p. 274-84.

122. Croitoru-Lamoury, I., et al., Interferon-gamma regulates the proliferation and differentiation of mesenchymal stem cells via activation of indoleamine 2,3 dioxygenase (IDO). PLoS One, 2011. 6(2): p. e14698.

123. Tu, Z., et al., Mesenchymal stem cells inhibit complement activation by secreting factor H. Stem Cells Dev, 2010. 19(11): p. 1803-9.

124. Ryan, J. M., et al., Interferon-gamma does not break, but promotes the immunosuppressive capacity of adult human mesenchymal stem cells. Clin Exp linmunol, 2007. 149(2): p. 353-63.

125. Noone, C., et al., IFN-gamma Stimulated Human Umbilical-Tissue-Derived Cells Potently Suppress NK Activation and Resist NK-Mediated Cytotoxicily In Vitro. Stem Cells Dev, 2013. 22(22): p. 3003-14.

126. Kwon, Y. W., et al., Tumor necrosis factor-alpha-activated mesenchymal stem cells promote endothelial progenitor cell homing and angiogenesis. Biochim Biophys Acta, 2013. 1832(12): p. 2136-44.

127. Lu, Z., et al., Activation and promotion of adipose stem cells by tumour necrosis factor-alpha preconditioning for bone regeneration. J Cell Physiol, 2013. 228(8): p. 1737-44.

128. Grote, K., et al., Toll-like receptor 2/6-dependent stimulation of mesenchymal stem cells promotes angiogenesis by paracrine factors. Eur Cell Mater, 2013. 26: p. 66-79; discussion 79.

129. Lalu, M. M., et al., Safety of cell therapy with mesenchymal stromal cells (SafeCell): a systematic review and meta-analysis of clinical trials. PLoS One, 2012. 7(10): p. e47559.

130. Mohyeddin Bonab, M., et al., Does mesenchymal stem cell therapy help multiple sclerosis patients? Report of a pilot study. fran I Immunol, 2007. 4(1): p. 50-7.

131. Yamout, B., et al., Bone marrow mesenchymal stem cell transplantation in patients with multiple sclerosis: a pilot study. J Neuroimmunol, 2010. 227(1-2): p. 185-9.

132. Bonab, M. M., et al., Autologous mesenchymal stem cell therapy in progressive multiple sclerosis: an open label study. Cuff Stem Cell Res Ther, 2012. 7(6): p. 407-14.

133. Karussis, D., et al., Safety and immunological effects of mesenchymal stem cell transplantation in patients with multiple sclerosis and amyotrophic lateral sclerosis. Arch Neurol, 2010. 67(10): p. 1187-94.

134. Maccario, R., et al., Interaction of human mesenchymal stem cells with cells involved in alloantigen-specific immune response favors the differentiation of CD4+ T-cell subsets expressing a regulatory/suppressive phenotype. Haematologica, 2005. 90(4): p. 516-25.

135. Prevosto, C., et al., Generation of CD4+ or CD8+ regulatory T cells upon mesenchymal stem cell-lymphocyte interaction. Haematologica, 2007. 92(7): p. 88 1-8.

136. Di Tanni, M., et al., Mesenchymal cells recruit and regulate T regulatory cells. Exp Hematol, 2008. 36(3): p. 309-18.

137. Casiraghi, F., et al., Pretransplant infusion of mesenchymal stem cells prolongs the survival of a semiallogeneic heart transplant through the generation of regulatory T cells. I Immunol, 2008. 181(6): p. 3933-46.

138. Boumaza, I., et al., Autologous bone marrow-derived rat mesenchymal stem cells promote PDX-1 and insulin expression in the islets, alter T cell cytokine pattern and preserve regulatory T cells in the periphery and induce sustained normoglycemia. J Autoimmun, 2009. 32(1): p. 33-42.

139. Ye, Z., et al., Immunosuppressive effects of rat mesenchymal stem cells: involvement of CD4+CD25+ regulatory T cells. Hepatobiliary Pancreat Dis Tnt, 2008. 7(6): p. 608-14.

140. Madec, A. M., et al., Mesenchymal stem cells protect NOD mice from diabetes by inducing regulatory T cells. Diabetologia, 2009. 52(7): p. 1391-9.

141. Mohajeri, M., et al., FOXP3 gene expression in multiple sclerosis patients pre- and post mesenchymal stem cell therapy. fran I Allergy Asthma Tmmunol, 2011. 10(3): p. 155-61.

142. Connick, P., et al., Autologous mesenchymal stem cells for the treatment of secondary progressive multiple sclerosis: an open-label phase 2a proof-of-concept study. Lancet Neurol, 2012. 11(2): p. 150-6.

143. Duijvestein, M., et al., Autologous bone marrow-derived mesenchymal stromal cell treatment for refractory luminal Crohn disease: results of a phase I study. Gut, 2010. 59(12): p. 1662-9.

144. Forbes, G. M., et al., A Phase 2 Study of Allogeneic Mesenchymal Stromal Cells for Luminal Crohn's Disease Refractory to Biologic Therapy. Clin Gastroenterol Hepatol, 2013.

145. Sun, L., et al., Mesenchymal stem cell transplantation reverses multiorgan dysfunction in systemic lupus erythematosus mice and humans. Stem Cells, 2009. 27(6): p. 1421-32.

146. Wang, D., et al., Double allogenic mesenchymal stem cells transplantations could not enhance therapeutic effect compared with single transplantation in systemic lupus erythematosus. Clin Dev Immunol, 2012. 2012: p. 273291.

147. Sayegh, M. H. and C. B. Carpenter, Transplantation 50 years later—progress, challenges, and promises. N Engl J Med, 2004. 351(26): p. 2761-6.

148. Sayegh, M. H. and G. Remuzzi, Clinical update: immunosuppression minimisation. Lancet, 2007. 369(9574): p. 1676-8.

149. Kawai, T., et al., HLA-mismatched renal transplantation without maintenance immunosuppression. N Engl J Med, 2008. 358(4): p. 353-61.

150. Perico, N., et al., Autologous mesenchymal stromal cells and kidney transplantation: a pilot study of safely and clinical feasibility. Clin J Am Soc Nephrol, 2011. 6(2): p. 412-22.

151. Reinders, M. E., et al., Autologous bone marrow-derived mesenchymal stromal cells for the treatment of allograft rejection after renal transplantation: results of a phase I study. Stem Cells Transl Med, 2013. 2(2): p. 107-11.

152. Tan, J., et al., Induction therapy with autologous mesenchymal stem cells in living-related kidney transplants: a randomized controlled trial. JAMA, 2012. 307(11): p. 1169-77.

153. Steinman, R. M., D. Hawiger, and M. C. Nussenzweig, Tolerogenic dendritic cells. Annu Rev Immunol, 2003. 21: p. 685-711.

154. Adema, G. J., Dendritic cells from bench to bedside and back. Immunol Lett, 2009. 122(2): p. 128-30.

155. Steinman, R. M. and K. Inaba, Myeloid dendritic cells. J Leukoc Biol, 1999. 66(2): p. 205-8.

156. Steinman, R.M., Dendritic cells and the control of immunity: enhancing the efficiency of antigen presentation. Mt Sinai J Med, 2001. 68(3): p. 160-6.

157. Bonifaz, L., et al., Efficient targeting of protein antigen to the dendritic cell receptor DEC-205 in the steady state leads to antigen presentation on major histocompatibilily complex class I products and peripheral CD8+T cell tolerance. J Exp Med, 2002. 196(12): p. 1627-38.

158. Mukhopadhaya, A., et al., Selective delivery of beta cell antigen to dendritic cells in vivo leads to deletion and tolerance of autoreactive CD8+ T cells in NOD mice. Proc Nati Acad Sci U S A, 2008. 105(17): p. 6374-9.

159. Giannoukakis, N., et al., Phase I (safety) study of autologous tolerogenic dendritic cells in type 1 diabetic patients. Diabetes Care, 2011. 34(9): p. 2026-32.

160. Krampera, M., et al., Bone marrow mesenchymal stem cells inhibit the response of naive and memory antigen-specific T cells to their cognate peptide. Blood, 2003. 101(9): p. 3722-9.

161. Zhang, W., et al., Effects of mesenchymal stem cells on differentiation, maturation, and function of human monocyte-derived dendritic cells. Stem Cells Dev, 2004. 13(3): p. 263-71.

162. Beyth, S., et al., Human mesenchymal stem cells alter antigen-presenting cell maturation and induce T-cell unresponsiveness. Blood, 2005. 105(5): p. 2214-9.

163. hang, X.X., et al., Human mesenchymal stem cells inhibit differentiation and function of monocyte-derived dendritic cells. Blood, 2005. 105(10): p. 4120-6.

164. Fibbe, W. E., A. J. Nauta, and H. Roelofs, Modulation of immune responses by mesenchymal stem cells. Ann N Y Acad Sci, 2007. 1106: p. 272-8.

165. Djouad, F., et al., Mesenchymal stem cells inhibit the differentiation of dendritic cells through an interleukin-6-dependent mechanism. Stem Cells, 2007. 25(8): p. 2025- 32.

166. Jung, Y. J., et al., MSC-DC interactions: MSC inhibit maturation and migration of BM-derived DC. Cytotherapy, 2007. 9(5): p. 451-8.

167. Chen, L., et al., Effects of human mesenchymal stem cells on the differentiation of dendritic cells from CD34+cells. Stem Cells Dev, 2007. 16(5): p. 719-31.

168. Wang, Q., et al., Murine bone marrow mesenchymal stem cells cause mature dendritic cells to promote T-cell tolerance. Scand J Immunol, 2008. 68(6): p. 607-15.

169. Oh, J. Y., et al., Intravenous mesenchymal stem cells prevented rejection of allogeneic corneal transplants by aborting the early inflammatory response. Mol Ther, 2012. 20(11): p. 2143-52.

170. Li, Y.P., et al., Human mesenchymal stem cells license adult CD34+hemopoietic progenitor cells to differentiate into regulatory dendritic cells through activation of the Notch pathway. J Immunol, 2008. 180(3): p. 1598-608.

171. Chiesa, S., et al., Mesenchymal stem cells impair in vivo T-cell priming by dendritic cells. Proc Natl Acad Sci US A, 2011. 108(42): p. 17384-9.

172. English, K., F. P. Barry, and B. P. Mahon, Murine mesenchymal stem cells suppress dendritic cell migration, maturation and antigen presentation. Immunol Lett, 2008. 115(1): p. 50-8.

173. Li, H., et al., Mesenchymal stem cells alter migratory properly of T and dendritic cells to delay the development of murine lethal acute graft-versus-host disease. Stem Cells, 2008. 26(10): p. 253 1-41.

174. Opitz, C .A., et al., Toll-like receptor engagement enhances the immunosuppressive properties of human bone marrow-derived mesenchymal stem cells by inducing indoleamine-2, 3-dioxygenase-1 via interferon-beta and protein kinase R. Stem Cells, 2009. 27(4): p. 909-19.

175. Fan, H., et al., Pre-treatment with IL-1 beta enhances the efficacy of MSC transplantation in DSS-induced colitis. Cell Mol Immunol, 2012. 9(6): p. 473-81.

176. Duijvestein, M., et al., Pretreatment with interferon-gamma enhances the therapeutic activity of mesenchymal stromal cells in animal models of colitis. Stem Cells, 2011. 29(10): p. 1549-58.

177. Krampera, M., et al., Role for interferon-gamma in the immunomodulatory activity of human bone marrow mesenchymal stem cells. Stem Cells, 2006. 24(2): p. 386-98.

178. Polchert, D., et al., IFN-gamma activation of mesenchymal stem cells for treatment and prevention of graft versus host disease. Eur I Immunol, 2008. 38(6): p. 1745-55.

179. Groh, M. E., et al., Human mesenchymal stem cells require monocyte-mediated activation to suppress alloreactive T cells. Exp Hematol, 2005. 33(8): p. 928-34.

180. Bartholomew, A., et al., Mesenchymal stem cells suppress lymphocyte proliferation in vitro and prolong skin graft survival in vivo. Exp Hematol, 2002. 30(1): p. 42-8.

181. Beggs, K. J., et al., Immunologic consequences of multiple, high-dose administration of allogeneic mesenchymal stem cells to baboons. Cell Transplant, 2006. 15(8-9): p.711-21.

182. Tse, W. T., et al., Suppression of allogeneic T-cell proliferation by human marrow stromal cells: implications in transplantation. Transplantation, 2003. 75(3): p. 389-97.

183. Maitra, B., et al., Human mesenchymal stem cells support unrelated donor hematopoietic stem cells and suppress T-cell activation. Bone Marrow Transplant, 2004. 33(6): p. 597-604.

184. Rasmusson, I., et al., Mesenchymal stem cells inhibit the formation of cytotoxic T lymphocytes, but not activated cytotoxic T lymphocytes or natural killer cells. Transplantation, 2003. 76(8): p. 1208-13.

185. Angoulvant, D., et al., Human mesenchymal stem cells suppress induction of cytotoxic response to alloantigens. Biorheology, 2004. 41(3-4): p. 469-76.

186. Le Blanc, K., et al., Mesenchymal stem cells inhibit the expression of CD25 (interleukin-2 receptor) and CD38 on phytohaemagglutinin-activated lymphocytes. Scand J Immunol, 2004. 60(3): p. 307-15.

187. Glennie, S., et al., Bone marrow mesenchymal stem cells induce division arrest anergy of activated T cells. Blood, 2005. 105(7): p. 2821-7.

188. Kim, J. A., et al., The inhibition of T-cells proliferation by mouse mesenchymal stem cells through the induction of p16INK4A-cyclin D1/cdk4 and p21waf1, p27kip1-cyclin E/cdk2 pathway. Cell Immunol, 2007. 245(1): p. 16-23.

189. Zappia, E., et al., Mesenchymal stem cells ameliorate experimental autoimmune encephalomyelitis inducing T-cell anergy. Blood, 2005. 106(5): p. 1755-61.

190. Plumas, I., et al., Mesenchymal stem cells induce apoptosis of activated T cells. Leukemia, 2005. 19(9): p. 1597-604.

191. Lim, J.H., et al., Immunomodulation of delayed-type hypersensitivity responses by mesenchymal stem cells is associated with bystander T cell apoptosis in the draining lymph node. J Immunol, 2010. 185(7): p. 4022-9

192. Rasmusson, I., et al., Mesenchymal stem cells inhibit lymphocyte proliferation by mitogens and alloantigens by different mechanisms. Exp Cell Res, 2005. 305(1): p. 33-41.

193. Xu, G., et al., Immunosuppressive properties of cloned bone marrow mesenchymal stem cells. Cell Res, 2007. 17(3): p. 240-8.

194. English, K., et al., Cell contact, prostaglandin E(2) and transforming growth factor beta 1 play non-redundant roles in human mesenchymal stem cell induction of CD4+CD25(High)forkhead box P3+ regulatory T cells. Clin Exp Immunol, 2009. 156(1): p. 149-60.

195. Spaggiari, G. M., et al., MSCs inhibit monocyte-derived DC maturation and function by selectively interfering with the generation of immature DCs: central role of MSC-derived prostaglandin E2. Blood, 2009. 113(26): p. 6576-83.

196. Yanez, R., et al., Prostaglandin E2 plays a key role in the immunosuppressive properties of adipose and bone marrow tissue-derived mesenchymal stromal cells. Exp Cell Res, 2010. 316(19): p. 3 109-23.

197. Zafranskaya, M., et al., PGE2 Contributes to In vitro MSC-Mediated Inhibition of Non-Specific and Antigen-Specific T Cell Proliferation in MS Patients. Scand J Immunol, 2013. 78(5): p. 455-62.

198. Nasef, A., et al., Identification of IL-10 and TGF-beta transcripts involved in the inhibition of T-lymphocyte proliferation during cell contact with human mesenchymal stem cells. Gene Expr, 2007. 13(4-5): p. 217-26.

199. Magatti, M., et al., Human amnion mesenchyme harbors cells with allogeneic T-cell suppression and stimulation capabilities. Stem Cells, 2008. 26(1): p. 182-92.

200. El Haddad, N., et al., Mesenchymal stem cells express serine protease inhibitor to evade the host immune response. Blood, 2011. 117(4): p. 1176-83.

201. Sato, K., et al., Nitric oxide plays a critical role in suppression of T-cell proliferation by mesenchymal stem cells. Blood, 2007. 109(1): p. 228-34.

202. Oh, I., et al., Interferon-gamma and NF-kappaB mediate nitric oxide production by mesenchymal stromal cells. Biochem Biophys Res Commun, 2007. 355(4): p. 956-62.

203. Ren, G., et al., Mesenchymal stem cell-mediated immunosuppression occurs via concerted action of chemokines and nitric oxide. Cell Stem Cell, 2008. 2(2): p. 141-50.

204. DelaRosa, O., et al., Requirement of IFN-gamma-mediated indoleamine 2,3-dioxygenase expression in the modulation of lymphocyte proliferation by human adipose-derived stem cells. Tissue Eng Part A, 2009. 15(10): p. 2795-806.

205. Tipnis, S., C. Viswanathan, and A. S. Majumdar, Immunosuppressive properties of human umbilical cord-derived mesenchymal stem cells: role of B 7-H1 and IDO. Immunol Cell Biol, 2010. 88(8): p. 795-806.

206. Ge, W., et al., Regulatory T-cell generation and kidney allograft tolerance induced by mesenchymal stem cells associated with indoleamine 2,3-dioxygenase expression. Transplantation, 2010. 90(12): p. 13 12-20.

207. Francois, M., et al., Human MSC suppression correlates with cytokine induction of indoleamine 2,3-dioxygenase and bystander M2 macrophage differentiation. Mol Ther, 2012. 20(1): p. 187-95.

208. Sattler, C., et al., Inhibition of T-cell proliferation by murine multipotent mesenchymal stromal cells is mediated by CD39 expression and adenosine generation. Cell Transplant, 2011. 20(8): p. 1221-30.

209. Saldanha-Araujo, F., et al., Mesenchymal stromal cells up-regulate CD39 and increase adenosine production to suppress activated T-lymphocytes. Stem Cell Res, 2011. 7(1): p. 66-74.

210. Barry, F., et al., The SH-3 and SH-4 antibodies recognize distinct epitopes on CD 73 from human mesenchymal stem cells. Biochem Biophys Res Commun, 2001. 289(2): p. 5 19-24.

211. Xue, Q., et al., The negative co-signaling molecule b7-h4 is expressed by human bone marrow-derived mesenchymal stem cells and mediates its T-cell modulatory activity. Stem Cells Dev, 2010. 19(1): p. 27-38.

212. Gieseke, F., et al., Human multipotent mesenchymal stromal cells use galectin-1 to inhibit immune effector cells. Blood, 2010. 116(19): p. 3770-9.

213. Chabannes, D., et al., A role for heme oxygenase-1 in the immunosuppressive effect of adult rat and human mesenchymal stem cells. Blood, 2007. 110(10): p. 369 1-4.

214. Mougiakakos, D., et al., The impact of inflammatory licensing on heme oxygenase-1-mediated induction of regulatory T cells by human mesenchymal stem cells. Blood, 2011. 117(18): p. 4826-35.

215. Augello, A., et al., Bone marrow mesenchymal progenitor cells inhibit lymphocyte proliferation by activation of the programmed death 1 pathway. Eur J Immunol, 2005. 35(5): p. 1482-90.

216. Sheng, H., et al., A critical role of IFNgamma in priming MSC-mediated suppression of T cell proliferation through up-regulation of B7-H1. Cell Res, 2008. 18(8): p. 846-57.

217. Luz-Crawford, P., et al., Mesenchymal stem cells repress Th17 molecular program through the PD-1 pathway. PLoS One, 2012. 7(9): p. e45272.

218. Akiyama, K., et al., Mesenchymal-stem-cell-induced immunoregulation involves FAS-ligand-FAS-mediated T cell apoptosis. Cell Stem Cell, 2012. 10(5): p. 544-55.

219. Najar, M., et al., Characterization and functionality of the CD200-CD200R system during mesenchymal stromal cell interactions with T-lymphocytes. Immunol Lett, 2012. 146(1-2): p. 50-6.

220. Batten, P., et al., Human mesenchymal stem cells induce T cell anergy and downregulate T cell allo-responses via the TH2 pathway: relevance to tissue engineering human heart valves. Tissue Eng, 2006. 12(8): p. 2263-73.

221. Lu, X., et al., Immunomodulatory effects of mesenchymal stem cells involved in favoring type 2 T cell subsets. Transpl Immunol, 2009. 22(1-2): p. 55-61.

222. Ko, E., et al., Mesenchymal stem cells inhibit the differentiation of CD4+ T cells into interleukin-17-secreting T cells. Acta Haematol, 2008. 120(3): p. 165-7.

223. Rafei, M., et al., Mesenchymal stromal cells ameliorate experimental autoimmune encephalomyelitis by inhibiting CD4 Th17 T cells in a CC chemokine ligand 2-dependent manner. J Immunol, 2009. 182(10): p. 5994-6002.

224. Tatara, R., et al., Mesenchymal stromal cells inhibit Th17 but not regulatory T-cell differentiation. Cytotherapy, 2011. 13(6): p. 686-94.

225. Duffy, M .M., et al., Mesenchymal stem cell inhibition of T-helper 17 cell-differentiation is triggered by cell-cell contact and mediated by prostaglandin E2 via the EP4 receptor. Eur J Immunol, 2011. 41(10): p. 2840-51.

226. Luz-Crawford, P., et al., Mesenchymal stem cells generate a CD4+CD25+Foxp3+regulatory T cell population during the differentiation process of Th1 and Th17 cells. Stem Cell Res Ther, 2013. 4(3): p. 65.

227. Kota, D. J., et al., TSG-6 produced by hMSCs delays the onset of autoimmune diabetes by suppressing Th1 development and enhancing tolerogenicily. Diabetes, 2013. 62(6): p. 2048-58.

228. Del Papa, B., et al., Notch1 modulates mesenchymal stem cells mediated regulatory T-cell induction. Eur I Immunol, 2013. 43(1): p. 182-7.

229. Zhang, X., et al., Generation of therapeutic dendritic cells and regulatory T cells for preventing allogeneic cardiac graft rejection. Clin Immunol, 2008. 127(3): p. 3 13-21.

230. Ichim, T. E., R. Zhong, and W. P. Min, Prevention of allo graft rejection by in vitro generated tolerogenic dendritic cells. Transpl Immunol, 2003. 11(3-4): p. 295-306.

231. Tiemessen, M. M., et al., CD4+CD25+Foxp3+ regulatory T cells induce alternative activation of human monocytes/macrophages. Proc Natl Acad Sci U S A, 2007. 104(49): p. 19446-51.

232. Gonzalez-Rey, E., et al., Human adipose-derived mesenchymal stem cells reduce inflammatory and T-cell responses and induce regulatory T cells in vitro in rheumatoid arthritis. Ann Rheum Dis, 2009.

233. Chang, C. J., et al., Placenta-derived multipotent cells exhibit immunosuppressive properties that are enhanced in the presence of interferon-gamma. Stem Cells, 2006. 24(11): p. 2466-77.

234. Li, C., et al., Human-placenta-derived mesenchymal stem cells inhibit proliferation and function of allogeneic immune cells. Cell Tissue Res, 2007. 330(3): p. 437-46.

235. Niemeyer, P., et al., Comparison of immunological properties of bone marrow stromal cells and adipose tissue-derived stem cells before and after osteogenic differentiation in vitro. Tissue Eng, 2007. 13(1): p. 111-21.

236. Wan, C. D., et al., Immunomodulatory effects of mesenchymal stem cells derived from adipose tissues in a rat orthotopic liver transplantation model. Hepatobiliary Pancreat Dis Tnt, 2008. 7(1): p. 29-33.

237. Gonzalez-Rey, E., et al., Human adipose-derived mesenchymal stem cells reduce inflammatory and T cell responses and induce regulatory T cells in vitro in rheumatoid arthritis. Ann Rheum Dis, 2010. 69(1): p. 241-8.

238. Gonzalez-Rey, E., et al., Human adult stem cells derived from adipose tissue protect against experimental colitis and sepsis. Gut, 2009. 58(7): p. 929-39.

239. Bai, L., et al., Human bone marrow-derived mesenchymal stem cells induce Th2-polarized immune response and promote endogenous repair in animal models of multiple sclerosis. Glia, 2009. 57(11): p. 1192-203.

240. Tisato, V., et al., Mesenchymal stem cells of cord blood origin are effective at preventing but not treating graft-versus-host disease. Leukemia, 2007. 21(9): p. 1992-9.

241. Keyser, K. A., K. E. Beagles, and H. P. Kiem, Comparison of mesenchymal stem cells from different tissues to suppress T-cell activation. Cell Transplant, 2007. 16(5): p. 555-62.

242. Magatti, M., et al., Amniotic mesenchymal tissue cells inhibit dendritic cell differentiation of peripheral blood and amnion resident monocytes. Cell Transplant, 2009. 18(8): p. 899-914.

243. Weiss, M. L., et al., Immune properties of human umbilical cord Wharton's jelly-derived cells. Stem Cells, 2008. 26(11): p. 2865-74.

244. Cutler, A. J., et al., Umbilical cord-derived mesenchymal stromal cells modulate monocyte function to suppress T cell proliferation. I Tmmunol, 2010. 185(11): p. 6617-23.

245. Corcione, A., et al., Human mesenchymal stem cells modulate B-cell functions. Blood, 2006. 107(1): p. 367-72.

246. Comoli, P., et al., Human mesenchymal stem cells inhibit antibody production induced in vitro by allostimulation. Nephrol Dial Transplant, 2008. 23(4): p. 1196-202.

247. Tabera, S., et al., The effect of mesenchymal stem cells on the viability, proliferation and differentiation of B-lymphocytes. Haematologica, 2008. 93(9): p. 1301-9.

248. Rafei, M., et al., Mesenchymal stromal cell-derived CCL2 suppresses plasma cell immuno globulin production via STAT3 inactivation and PAX5 induction. Blood, 2008. 112(13): p. 4991-8.

249. Asari, S., et al., Mesenchymal stem cells suppress B-cell terminal differentiation. Exp Hematol, 2009. 37(5): p. 604-15.

250. Franquesa, M., et al., Immunomodulatory effect of mesenchymal stem cells on B cells. Front Immunol, 2012. 3: p. 212.

251. Ma, X., et al., Allogenic mesenchymal stem cell transplantation ameliorates nephritis in lupus mice via inhibition of B-cell activation. Cell Transplant, 2012.

252. Schena, F., et al., Interferon-gamma-dependent inhibition of B cell activation by bone marrow-derived mesenchymal stem cells in a murine model of systemic lupus erythematosus. Arthritis Rheum, 2010. 62(9): p. 2776-86.

253. Guo, Y., et al., Human mesenchymal stem cells upregulate CD1dCD5(+) regulatory B cells in experimental autoimmune encephalomyelitis. Neuroimmunomodulation, 2013. 20(5): p. 294-303.

254. Bochev, I., et al., Mesenchymal stem cells from human bone marrow or adipose tissue differently modulate mitogen-stimulated B-cell immuno globulin production in vitro. Cell Biol Int, 2008. 32(4): p. 3 84-93.

255. Saka, Y., et al., Adipose-derived stromal cells cultured in a low-serum medium, but not bone marrow-derived stromal cells, impede xenoantibody production. Xenotransplantation, 2011. 18(3): p. 196-208.

256. Che, N., et al., Umbilical cord mesenchymal stem cells suppress B-cell proliferation and differentiation. Cell Immunol, 2012. 274(1-2): p. 46-53.

257. Liu, O., et al., Periodontal ligament stem cells regulate B lymphocyte function via programmed cell death protein 1. Stem Cells, 2013. 31(7): p. 1371-82.

Each of the above-listed references and all other references mentioned or cited in the present application are hereby incorporated by reference, each in its entirety.

1. A method of treatment autoimmunity comprising the steps of: a) obtaining a mesenchymal stem cell population; b) transfecting said mesenchymal stem cell population with one or more autoantigens of interest; c) administering said MSC into a patient in need of therapy.

2. The method of claim 1, wherein said mesenchymal stem cell is a Wharton's Jelly derived mesenchymal stem cell.

3. The method of claim 2, wherein said Wharton's jelly MSC expresses markers selected from a group comprising of CD73, CD105, CD90 and lacks expression of CD14, CD34 and CD45.

4. The method of claim 1, wherein said antigen is selected from a group comprising of:
myelin oligodendrocyte protein; b) myelin basic protein; c) collagen II; and d) myofibril protein.

5. The method of claim 1 wherein said transfection is performed by means of a plasmid DNA.

6. The method of claim 1, wherein said transfection is performed by lentivirus.

7. The method of claim 1, wherein said transfection is performed by adenovirus.
